(19) United States
(12) Patent Application Publication
(10) Pub. No.: US 2022/0105144 A1
(43) Pub. Date: Apr. 7, 2022

(54) PHARMACEUTICAL COMPOSITION FOR PREVENTING OR TREATING MALE SEXUAL DYSFUNCTION

(71) Applicant: WELLNESSBIO INC., Seoul (KR)

(72) Inventors: Si-young CHANG, Suwon-si (KR); Eun-ai CHO, Seoul (KR)

(73) Assignee: WELLNESSBIO INC., Seoul (KR)

(21) Appl. No.: 17/418,990

(22) PCT Filed: Dec. 18, 2019
(86) PCT No.: PCT/KR2019/017959

§ 371 Date: Jun. 28, 2021

(30) Foreign Application Priority Data
Dec. 28, 2018 (KR) 10-2018-0172968

(51) Int. Cl. A61K 36/15; A61K 47/69; A61P 15/10

(52) U.S. Cl. CPC A61K 36/15; A61K 47/6951; A61P 15/10; A61K 2236/55; A61K 2236/331; A61K 2236/37; A61K 2236/31

(57) ABSTRACT

The present invention relates to a composition and health functional food for preventing or treating male sexual dysfunction, which include, as an active ingredient, oil separated from a Pinus densiflora extract, oil separated from an Abies holophylla Maxim extract, or a mixture thereof. The composition according to the present invention has an excellent ability to enhance penile erectility, and may be usefully applied to a health food for alleviating erectile dysfunction symptoms or a therapeutic agent for erectile dysfunction.

### TECHNICAL FIELD

### Cross-Reference to Related Application(s)

This application claims the benefit of Korean Patent Application No. 10-2018-0172968, filed on Dec. 28, 2018, in the Korean Intellectual Property Office, the disclosure of which is incorporated herein in its entirety by reference.

The present invention relates to a pharmaceutical composition for preventing or treating male sexual dysfunction, which includes, as an active ingredient, oil separated from a Pinus densiflora extract, oil separated from an Abies holophylla Maxim extract, or a mixture thereof.

### BACKGROUND ART

Erectile dysfunction is a phenomenon in which blood is unable to normally flow into the corpus cavernosum tissue and refers to the case of an insufficient penile erection or non-persistent erection which accounts for 25% or more of the whole sexual life. The causes of erectile dysfunction are broadly classified into psychogenic and organic, and organic causes are divided into neurological, endocrine, vascular, and systemic diseases (Non-Patent Document 1. KIM, Se Chul. Diagnosis and Treatment of Male Sexual Dysfunction. ILJOGAK; 1995 36-162).

Among these, neuronal erectile dysfunction is caused by brain tumors, cerebrovascular diseases, spinal cord injury, diabetes, peripheral neuropathy due to chronic alcoholism (Non-Patent Document 2, Benet A E, Melman A. The epidemiology of erectile dysfunction. Urol Clin North Am. 1995; 22(4): 699-709).

Generally, an erection can be easily achieved when blood vessels in the corpus cavernosum tissues are relaxed and blood flows smoothly. Vascular relaxation reactions are known to be caused by vascular endothelial relaxation factors, which are a type of hormonal substance present in vascular endothelial cells, i.e., nitric oxide (NO) (Non-Patent Document 3, Feelisch M, Noack E. Nitric oxide (NO) formation from nitrovasodilators occurs independently of hemoglobin or non-heme iron. Eur J Pharmacol. 1987; 142: 465-469).

Nitric oxide (NO) is a non-adrenergic non-cholinergic (NANC) potent neuro-transmitter in the central and peripheral nervous systems, which is biosynthesized by nitric oxide synthase (NOS)(Patent Document 4, Bredt D S, Ferris C D, Snyder S H. Nitric oxide synthase regulatory sites. J Biol Chem. 1992; 267(16): 1976-1981).

Nitric oxide (NO) promotes the biosynthesis of the vasodilator cyclic-GMP (c-GMP) through guanylate cyclase activation in the corpus cavernosum, thus enabling an erection (Non-Patent Document 5, McMahon C G, Samali R, Johnson H. Efficacy, safety and patient acceptance of sildenafil citrate as treatment for erectile dysfunction. J Urol. 2000; 164: 1192-1196).

Since cyclic-GMP is generally degraded by phosphodiesterase (PDE), it is necessary to suppress the activity of phosphodiesterase for persistent erectile function (Non-Patent Document 6, Lincoln T M. Cyclic GMP and mechanism of vasodilation. Pharmacol Ther. 1989; 41: 479-502).

As a drug for the treatment of erectile dysfunction, sildenafil, which is an oral therapeutic agent, was developed in the 1990s and commercialized, and various products such as tadalafil, vardenafil, and the like were sold. These drugs are known to inhibit the action of phosphodiesterase-5 (PDE-5), which is an enzyme that returns the erect penis to its original state.

However, PDE-5 inhibitors are present in the gastrointestinal tract, nose, face, cerebral blood vessels, cardiac blood vessels, and the like, in addition to the penis, and may cause side effects when acting on areas other than the penis, and may also cause digestive disorders or headaches. In addition, patients who take organic nitrate have side effects such as a sharp drop in blood pressure when taking PDE-5 inhibitors.

Therefore, there is a need to develop novel formulations capable of preventing or treating erectile dysfunction while addressing the above-described problems.

### REFERENCES

### Non-Patent Documents

(Non-Patent Document 1) KIM, Se Chul. Diagnosis and Treatment of Male Sexual Dysfunction. ILJOGAK; 1995 36-162.
(Non-Patent Document 2) Benet A E, Melman A. The epidemiology of erectile dysfunction. Urol Clin North Am. 1995; 22(4): 699-709.
(Non-Patent Document 3) Feelisch M, Noack E. Nitric oxide (NO) formation from nitrovasodilators occurs independently of hemoglobin or non-heme iron. Eur J Pharmacol. 1987; 142: 465-469.
(Non-Patent Document 4) Bredt D S, Ferris C D, Snyder S H. Nitric oxide synthase regulatory sites. J Biol Chem. 1992; 267(16):1976-1981.
(Non-Patent Document 5) McMahon C G, Samali R, Johnson H. Efficacy, safety and patient acceptance of sildenafil citrate as treatment for erectile dysfunction. J Urol. 2000; 164: 1192-1196.
(Non-Patent Document 6) Lincoln T M. Cyclic GMP and mechanism of vasodilation. Pharmacol Ther. 1989; 41:479-502.

### DISCLOSURE OF INVENTION

### Technical Problem

An object of the present invention is to provide a pharmaceutical composition for preventing or treating male sexual dysfunction, which includes, as an active ingredient, oil separated from a Pinus densiflora extract, oil separated from an Abies holophylla Maxim extract, or a mixture thereof.

Another object of the present invention is to provide a health functional food for preventing or treating male sexual dysfunction, which includes, as an active ingredient, oil separated from a Pinus densiflora extract, oil separated from an Abies holophylla Maxim extract, or a mixture thereof.

Another object of the present invention is to provide a formulation including: oil separated from a Pinus densiflora extract, oil separated from an Abies holophylla Maxim extract, or a mixture thereof; and β-cyclodextrin.

Another object of the present invention is to provide a health functional food including: oil separated from a Pinus densiflora extract, oil separated from an Abies holophylla Maxim extract, or a mixture thereof; and 3-cyclodextrin.

### Solution to Problem

To address the above-described problems, according to the present invention, a pharmaceutical composition for preventing or treating male sexual dysfunction, including, as an active ingredient, oil separated from a Pinus densiflora extract, oil separated from an Abies holophylla Maxim extract, or a mixture thereof, which are natural substances, was developed, and it was confirmed that the composition exhibited a relaxation effect in the corpus cavernosum of white mice in in-vitro experiments and a synergistic effect on penile erectility when orally administered to white mice in in-vivo experiments, thus exhibiting a significant activity in the prevention or treatment of sexual dysfunction.

Hereinafter, the present invention will be described in detail.

Pharmaceutical Composition for Preventing or Treating Male Sexual Dysfunction

The present invention provides a pharmaceutical composition for preventing or treating male sexual dysfunction, which includes, as an active ingredient, oil separated from a Pinus densiflora extract, oil separated from an Abies holophylla Maxim extract, or a mixture thereof.

Since the composition according to the present invention has an effect of preventing or treating male sexual dysfunction, the composition may be usefully applied to an erectile dysfunction therapeutic agent or a health food for alleviating erectile dysfunction symptoms.

Specifically, as a result of conducting an experiment for measuring the content of nitrite (NO₂), an experiment for measuring the activity of nitric oxide (NO) synthase, an experiment for measuring the activity of guanyl cyclase, and an experiment for measuring the content of cyclic GMP, the composition according to the present invention may exhibit a significant increase in content or activity compared to a control, may exhibit a synergistic effect on penile erectility when orally administered to white mice in in-vivo experiments, and may also be anticipated to exhibit a relaxation effect in the corpus cavernosum of white mice in in-vitro experiments.

In addition, the inhibitory effects of PDE-5 enzymes of tadalafil and vardenafil, which are therapeutic agents for erectile dysfunction, also affect the gastrointestinal tract, nose, face, cerebral blood vessels, cardiac blood vessels, and the like, in addition to the smooth muscles of the prostate and bladder, and thus side effects such as backaches (tadalafil) and electrocardiogram abnormalities (vardenafil) frequently occur. However, since the composition of the present invention includes, as an active ingredient, oil separated from a Pinus densiflora extract, oil separated from an Abies holophylla Maxim extract, or a mixture thereof, which are natural substances, side effects, which are caused when the PDE-5 inhibitors, i.e., tadalafil and vardenafil, are used, do not occur.

In addition, the use of PDE-5 inhibitors such as tadalafil and vardenafil is contraindicated in patients who are taking organic nitrate formulations, but since the composition of the present invention includes, as an active ingredient, oil separated from a Pinus densiflora extract, oil separated from an Abies holophylla Maxim extract, or a mixture thereof, which are natural substances, it is possible for patients who are taking organic nitrate formulations to also take the composition of the present invention.

In the present invention, the term “Pinus densiflora” refers to an evergreen tree of the family Pinaceae, and in oriental medicines, pine bark, pine cones, pine resin, and pine needles are known to have efficacy against neuralgia, tonicity, gonorrhea, rheumatism, fracture pain, arthritis, stomatitis, cerebral hemorrhage, arteriosclerosis, headaches, edema, insomnia, car sickness, fatigue recovery, and the like, and oil obtained by distilling pine needles contains components such as 3-carene, limonene, terpinolene, and the like.

In the present invention, the term “Abies holophylla Maxim” also refers to an evergreen tree of the family Pinaceae like Pinus densiflora, and oil obtained by distilling the leaves thereof contains components such as α-pinene, β-pinene, camphene, terpineol, bornyl acetate, citral, and the like.

In the present invention, the term “oil” means any substance that is completely or partially liquid at about 10° C. to about 40° C. and is hydrophobic but is soluble in at least one organic solvent, and may be referred to as “an essential oil extract”. The term “about” represents a numerical value that is ±10% of the value it precedes.

In the present invention, the term “mixture thereof” is intended to include a case where Pinus densiflora and Abies holophylla Maxim are mixed and extracted, and then oil is obtained from the resulting extract, a case where Pinus densiflora and Abies holophylla Maxim are mixed, and then oil is obtained from the resulting mixture, or a case where oil obtained from a Pinus densiflora extract and oil obtained from an Abies holophylla Maxim extract are mixed, but the present invention is not limited thereto.

In the present invention, the term “active ingredient” refers to a component that can exhibit desired activity alone or can exhibit activity in combination with a carrier which is itself inactive.

In the present invention, the term “prevention” means inhibiting or delaying the onset of a disease, disorder, or illness. In the case where the onset of a disease, disorder, or illness is inhibited or delayed for a predetermined period of time, prevention may be considered complete.

In the present invention, the term “treatment” means partially or completely alleviating, ameliorating, relieving, inhibiting, or delaying a specific disease, disorder, and/or illness, or symptoms according to the illness, and reducing the severity thereof, or reducing the occurrence of one or more symptoms or features thereof.

In one embodiment of the present invention, the Pinus densiflora may mean pine branch and needle portions, and the Abies holophylla Maxim may mean fir branch and leaf portions. Specifically, the Pinus densiflora and the Abies holophylla Maxim is preferably understood as pine needles and fir needles. For example, the pinus densiflora extract or the Abies holophylla Maxim extract may specifically mean a pine needle extract or a fir needle extract.

In the present invention, the term “oil separated from a Pinus densiflora extract” refers to oil separated and obtained from an oil layer present on the top of the Pinus densiflora extract and is referred to as pine needle oil. For example, when the Pinus densiflora extract is obtained in a liquid state, the oil component included in the Pinus densiflora extract is positioned at an upper portion thereof, and water, which is a hydrophilic component, is positioned at a lower portion thereof. In this regard, oil positioned at the upper portion may be separated and obtained through a separator by removing the hydrophilic component positioned at the lower portion, and the obtained oil may be filtered to remove impurities, thereby obtaining oil separated from the Pinus densiflora extract.

In the present invention, the term “oil separated from an Abies holophylla Maxim extract” refers to oil separated and obtained from an oil layer present on the top of the Abies holophylla Maxim extract and is referred to as fir needle oil. For example, when the Abies holophylla Maxim extract is obtained in a liquid state, the oil component included in the Abies holophylla Maxim extract is positioned at an upper portion thereof, and the hydrophilic component is positioned at a lower portion thereof. In this regard, oil positioned at the upper portion may be separated and obtained through a separator by removing the hydrophilic component positioned at the lower portion, and the obtained oil may be filtered to remove impurities, thereby obtaining oil separated from the Abies holophylla Maxim extract.

In one embodiment of the present invention, the pharmaceutical composition of the present invention may be formulated, using a pharmaceutically acceptable carrier, in a unit dosage form or may be prepared by being put into a multi-dose container, according to a method that may be easily carried out by one of ordinary skill in the art to which the present invention pertains.

In the present invention, the term “carrier” refers to a compound that facilitates the addition of a compound into a cell or tissue, and the term “pharmaceutically acceptable” refers to a composition that is physiologically acceptable and generally does not cause allergic responses or responses similar thereto such as gastrointestinal disorders and dizziness when administered to humans.

The pharmaceutically acceptable carrier, which is commonly used in formulation, may be lactose, dextrose, sucrose, sorbitol, mannitol, starch, acacia gum, calcium phosphate, alginates, gelatin, calcium silicate, micro-crystalline cellulose, polyvinylpyrrolidone, cellulose, water, syrup, methyl cellulose, methylhydroxy benzoate, propylhydroxy benzoate, talc, magnesium stearate, and mineral oil, but the present invention is not limited thereto.

The pharmaceutical composition of the present invention may further include, in addition to the above ingredients, additives such as a filler, an anti-coagulant, a lubricant, a wetting agent, a flavoring agent, an emulsifier, a preservative, and the like. In the present invention, the amount of the additive included in the pharmaceutical composition is not particularly limited and may be appropriately adjusted within an amount range used in general formulation.

The pharmaceutical composition of the present invention may be for oral administration. In the present invention, the term “oral administration” means administration of a substance prepared for the digestion of the active ingredient to the gastrointestinal tract for absorption. Non-limiting examples of the formulation for oral administration include tablets, troches, lozenges, aqueous suspensions, oily suspensions, prepared powders, granules, emulsions, hard capsules, soft capsules, syrups, and elixirs. To formulate the pharmaceutical composition of the present invention for oral administration, a binder such as lactose, saccharose, sorbitol, mannitol, starch, amylopectin, cellulose, gelatin, or the like; an excipient such as dicalcium phosphate or the like; a disintegrant such as corn starch, sweet potato starch, or the like; a lubricant such as magnesium stearate, calcium stearate, sodium stearyl fumarate, polyethylene glycol, or the like; or the like may be used, and a sweetener, a fragrance, syrup, or the like may also be used. Furthermore, in the case of capsules, a liquid carrier such as fatty oil, and the like may be further used in addition to the aforementioned materials.

In the present invention, the term “excipient” refers to any substance that is not a therapeutic agent, and is intended to be used as a carrier or medium for the delivery of a therapeutic agent or added to the pharmaceutical composition. Thus, the excipient enhances handling and storage properties or allows or promotes the formation of a unit dose of the composition.

In one embodiment of the present invention, the oil may be included in an amount of 0.1 wt % to 15 wt %, particularly 0.1 wt % to 10 wt % or 0.1 wt % to 5 wt %, with respect to a total weight of the composition, but the present invention is not limited thereto.

The oil may have a specific gravity of 0.8 g/cm³ to 0.99 g/cm³, particularly 0.85 g/cm³ to 0.95 g/cm³, but the present invention is not limited thereto. In the present invention, the term “specific gravity” refers to a ratio of weight to volume, and refers to a ratio of the mass of a substance occupying a certain volume at a certain temperature to the mass of a standard substance at the same temperature and the same volume. The unit of specific gravity is g/cm³.

In one embodiment of the present invention, the extract may be obtained by treating pine needles or fir needles by steam distillation, but the present invention is not limited thereto.

In the present invention, the term “steam distillation” refers to a method commonly used for the extraction of pure oil, and may be divided into a method of passing collected leaves through hot steam and a method of heating a material, which has not been mixed with water, together with water.

For the steam distillation used in the present invention, a method of passing collected leaves through hot steam, cooling the obtained steam to obtain an extract, and then obtaining essential oil floating on the top layer of the extract was used.

In one embodiment of the present invention, the oil may be obtained by the following processes, but the present invention is not limited thereto.

(S1) collecting pine needles, fir needles, or a mixture thereof; and

(S2) contacting the collected pine needles, fir needles, or mixture thereof with steam to obtain an extract.

In one embodiment of the present invention, the contacting process may be a process of injecting steam at 1 atm to 8 atm into a reactor including pine needles, fir needles, or a mixture thereof, and particularly, may be a process of injecting steam at 1 atm to 3 atm into the reactor, but the present invention is not limited thereto.

In one embodiment of the present invention, a process of collecting oil from the obtained extract may be further performed, but the present invention is not limited thereto.

As an example of the present invention, the oil may be obtained through: a first process of collecting pine needles or fir needles and then sorting the same; a second process of washing the selected pine needles or fir needles and then drying the same at 40° C. to 80° C. for 3 hours to 7 hours; a third process of performing steam distillation on the dried pine or fir needles at 1 atm to 8 atm for 1 hours to 5 hours; and a fourth process of subjecting the resulting product to filtration and purification. Specifically, in the third process, the steam distillation may be performed at 1 atm to 3 atm for 1 hour to 5 hours, but the present invention is not limited thereto.

In one embodiment of the present invention, the male sexual dysfunction may be, but is not limited to, erectile dysfunction, premature ejaculation, or prostatic hyperplasia, and in particular, means erectile dysfunction.

In the present invention, the term “erectile dysfunction” refers to a state in which an erection is not sufficient or not maintained for sexual life, and is generally defined as erectile dysfunction when such a state lasts for 3 months or longer.

In one embodiment of the present invention, the male sexual dysfunction may be caused by ingestion of alcohol, for example, an alcoholic drink.

The composition of the present invention may be used by being formulated into various forms such as: oral formulations, e.g., liquids, suspensions, powders, granules, tablets, capsules, pills, extracts, emulsions, syrups, aerosols, and the like; injections such as sterile injection solutions; and the like, and may be orally administered or administered via various routes including intravenous administration, intraperitoneal administration, subcutaneous administration, intrarectal administration, local administration, and the like.

Formulations according to the present invention include oil separated from a Pinus densiflora extract, oil separated from an Abies holophylla Maxim extract, or a mixture thereof, and β-cyclodextrin.

In one embodiment of the present invention, the β-cyclodextrin may be any one of 2,6-dimethyl-β-cyclodextrin, 2-hydroxyethyl-β-cyclodextrin, and 2-hydroxypropyl-β-cyclodextrin, but the present invention is not limited thereto.

In one embodiment of the present invention, formulations according to the present invention include a β-cyclodextrin inclusion compound. The inclusion compound may be in a form in which oil separated from a Pinus densiflora extract, oil separated from an Abies holophylla Maxim extract, or a mixture thereof is encapsulated in the internal cavity of β-cyclodextrin.

In one embodiment of the present invention, preparations according to the present invention include the pharmaceutically acceptable carrier, a detailed description of the carrier is the same as provided above.

In the present invention, the term “liquid” means a medicine to be taken in the form of a portion dissolved in water or an organic solvent. The liquid has an advantage of more effective absorption of a drug into the systemic circulation in the intestinal tract compared to suspensions or solid preparations, and the liquid may also include an additional solute in addition to the drug and may also include an additive that imparts color, odor, sweetness, or stability.

In the present invention, the term “suspending agent” refers to any agonist capable of providing desired solubility and/or dispersibility of an alginate-containing composition, i.e., providing an aqueous formulation that is substantially transparent and free of sedimentation and lumps.

In the present invention, the term “powder” means a finely divided drug, a chemical, or a dried mixture of both.

In the present invention, the term “granule” refers to a granular form of a pharmaceutical or a mixture of medicines, which is generally within a range passing through a 4.76 mm to 20 mm sieve. Granules are generally produced by soaking a powder or powder mixture and passing the resulting mass through a sieve or granulator of a suitable mesh size depending on a desired size of granules. Granules are also in a particle state like powders, and thus a drug is highly likely to come into contact with the tongue, and therefore, when drugs with a bitter taste are used in a granular form, patients, especially children or the elderly may feel discomfort.

In the present invention, the term “tablet” means that a powdered medicine is compressed into a small disc shape to make it easy to take. Tablets may include uncoated tablets, film-coated tablets, sugar-coated tablets, multi-layered tablets, dry coated tablets, inner core tablets, orally disintegrating tablets, chewable tablets, effervescent tablets, dispersible tablets, soluble tablets, and the like.

In the present invention, the term “capsule” means that produced by filling capsules with a drug or encapsulation-molding a drug with a capsule base, in the form of liquid, a suspension, water, powder, a granule, a mini-tablet, a pellet, or the like.

In the present invention, the term “pill” is intended to encompass a small, round solid dosage form including composite particles mixed with a binder and other excipients.

In the present invention, the term “extract” refers to a semi-solid or solid formulation prepared by leaching an active ingredient in a vegetable or animal herbal medicine using a suitable leaching agent, evaporating the solvent to concentrate the active ingredient to a predetermined concentration, and adjusting the content of the concentrate by adding an excipient thereto when there are regulations on the contents of main ingredients.

In the present invention, the term “syrup” means a concentrated homemade product of sugar or a sugar substitute. In the present invention, the syrups are a formulation in which a medicine with an unpleasant taste, e.g., a bitter taste, is prepared into a liquid and that is easy to take, and particularly, is a formulation suitable for children to take. In the present invention, the syrups may include, in addition to purified water and an extract, tetrasaccharides, substitute drugs thereof used to impart sweetness and viscosity, antimicrobial preservatives, flavors, colorants, or the like, but the present invention is not limited thereto. Examples of sweeteners that may be included in such syrups include, but are not limited to, sucrose, mannitol, sorbitol, xylitol, aspartame, stevioside, fructose, lactose, sucralose, saccharin, and menthol.

A suitable dose of the pharmaceutical composition of the present invention may vary depending on the condition, body weight, age, and gender of a patient, health condition, dietary specificity, the nature of a formulation, the severity of disease, administration time of the composition, administration method, administration period or interval, excretion rate, and drug form, and may be appropriately selected by those of ordinary skill in the art. For example, the suitable dose may range from about 0.1 mg/kg to 10,000 mg/kg, about 1 mg/kg to 8,000 mg/kg, about 5 mg/kg to 6,000 mg/kg, or about 10 mg/kg to 4,000 mg/kg, preferably about 50 mg/kg to about 2,000 mg/kg, but the present invention is not limited thereto, and the pharmaceutical composition may be administered in a single dose or divided into multiple doses daily.

In the present specification, the term “effective dosage of the pharmaceutical composition” means the amount of an active ingredient of the composition sufficient to treat specific symptoms. The effective dosage may vary depending on formulation method, administration method, administration time, and/or administration route, may vary according to several factors including the type and extent of a reaction to be achieved via administration of the pharmaceutical composition, the type, age, and body weight of a subject to which the pharmaceutical composition is to be administered, general health condition, the symptoms or severity of disease, gender, diet, excretion, ingredients of drugs and other compositions used simultaneously in the corresponding subject, and the like, and similar factors well known in the medical field, and the effective dosage suitable for desired treatment may be easily determined and prescribed by one of ordinary skill in the art.

The pharmaceutical composition of the present invention may be administered as a single dose or divided into multiple doses daily. The pharmaceutical composition of the present invention may be administered as an individual therapeutic agent or in combination with other therapeutic agents, and may be administered sequentially or simultaneously with conventional therapeutic agents. The pharmaceutical composition may be administered in the minimum amount that enables achievement of the maximum effects without side effects in consideration of all the above-described factors, and this may be easily determined by those of ordinary skill in the art to which the present invention pertains.

Health Functional Food for Preventing or Treating Male Sexual Dysfunction

The present invention provides a health functional food for preventing or treating male sexual dysfunction, which includes, as an active ingredient, oil separated from a Pinus densiflora extract, oil separated from an Abies holophylla Maxim extract, or a mixture thereof.

For the health functional food according to the present invention, oil separated from a Pinus densiflora extract, oil separated from an Abies holophylla Maxim extract, or a mixture thereof, according to the present invention, may be used alone or in combination with other foods or food ingredients, and may be appropriately used according to a general method.

A health food according to the present invention includes oil separated from a Pinus densiflora extract, oil separated from an Abies holophylla Maxim extract, or a mixture thereof, and β-cyclodextrin.

The inclusion compound may be in a form in which oil separated from a Pinus densiflora extract, oil separated from an Abies holophylla Maxim extract, or a mixture thereof is encapsulated in the internal cavity of 3-cyclodextrin.

In the present invention, the term “functional food” means a food produced and processed using raw materials or ingredients having functionality useful for the human body, and in the present invention, the functional food has a beneficial effect on improving sexual function. In the present invention, the term “functionality” may mean an effect useful for health use by controlling nutrients or physiological actions on the structure and function of the human body.

Oil separated from a Pinus densiflora extract, oil separated from an Abies holophylla Maxim extract, or a mixture thereof, which is an active ingredient of the health functional food of the present invention, may be used alone or in combination with other foods or other food ingredients, and may be appropriately used according to a general method.

The type of food is not particularly limited. Examples of foods to which the oil separated from a Pinus densiflora extract, oil separated from an Abies holophylla Maxim extract, or a mixture thereof may be added include various foods, beverages, types of gum, teas, candies, vitamin complexes, health functional foods, powders, granules, tablets, capsules, types of jelly, drinks, and the like, and may include all health foods in a general sense.

One of the oils or a mixture thereof may be added to a food or a beverage for the purpose of preventing and treating sexual function. In this regard, the amount of the extract in the food may range from 0.01 wt % to 30 wt % with respect to a total weight of the food, and the amount of the extract in the beverage composition may range from 0.01 wt % to 10 wt %, preferably 0.01 wt % to 5 wt %, with respect to 100 mL of the beverage composition, but the present invention is not limited thereto.

In addition, the oil, which is an active ingredient of the present invention, may be included in an amount of 0.1 wt % to 30 wt %, preferably 0.1 wt % to 10 wt %, and more preferably 0.1 wt % to 5 wt %, with respect to a total amount of raw materials, but the present invention is not limited thereto.

The food of the present invention has no particular limitation on other ingredients except for an ingredient including the oil separated from a Pinus densiflora extract, the oil separated from an Abies holophylla Maxim extract, or a mixture thereof, and may include various flavoring agents, natural carbohydrates, or the like as additives as in general beverages, but the present invention is not limited thereto. The natural carbohydrates include saccharides such as glucose, fructose, maltose, sucrose, dextrin, and cyclodextrin; and sugar alcohols such as xylitol, sorbitol, erythritol, and the like. As the flavoring agent, a natural flavoring agent (thaumatin and stevia extracts (e.g., rebaudioside A, glycyrrhizin, and the like)) and a synthetic flavoring agent (saccharin, aspartame, and the like) may be used, but the present invention is not limited thereto.

In addition, the food of the present invention may further include various nutritional supplements, vitamins, minerals (electrolytes), flavors such as synthetic flavors, natural flavors, and the like, colorants and enhancers, pectic acid and salts thereof, alginic acid, citric acid, sodium citrate and salts thereof, organic acids, a protective colloid thickener, a pH adjuster, a stabilizer, a preservative, glycerin, alcohol, a carbonating agent used in carbonated beverages, and the like, but the present invention is not limited thereto. These additives may generally be selected from an amount range of 0.1 part by weight to 50 parts by weight with respect to 1 part by weight of oil separated from a Pinus densiflora extract, an Abies holophylla Maxim extract, or a mixture thereof, which is an active ingredient, but the present invention is not limited thereto.

The details mentioned in the compositions, formulations, and health functional foods of the present invention are equally applied unless they contradict each other.

### Advantageous Effects of Invention

A composition according to the present invention has an excellent ability to increase penile erectility, and can be effectively applied as a health food for alleviating erectile dysfunction symptoms or a therapeutic agent for erectile dysfunction.

### BRIEF DESCRIPTION OF DRAWINGS

FIG. 1 is a schematic conceptual view illustrating a method of separating oil from an extract through steam distillation.

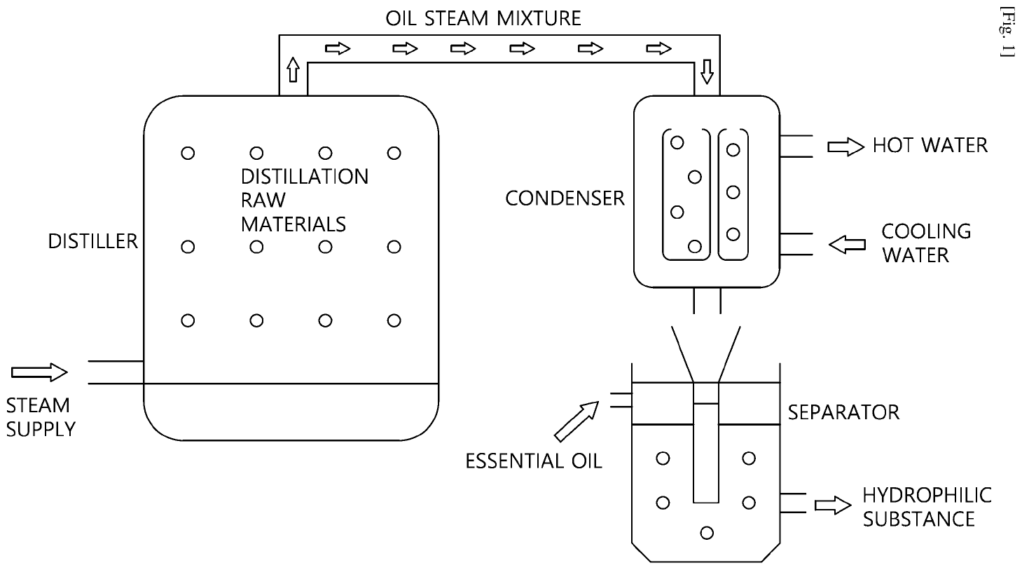

FIG. 2 is a graph showing the results of evaluating cyclic GMP content in corpus cavernosum smooth muscles.

### MODE FOR THE INVENTION

Hereinafter, configurations and effects of the present invention will be described in further detail with reference to the following examples. These examples are provided for illustrative purposes only and are not intended to limit the scope of the present invention.

In addition, reagents and solvents, which will be described below, were purchased from Sigma-Aldrich unless otherwise specifically mentioned herein.

### EXAMPLES

### <Example 1> Preparation of Oil Separated from Pinus Densiflora Extract

5 kg of pine needles (Collection place: Yeongcheon, Gyeongbuk) were taken, washed with 1.5 L of distilled water, dried in a dry oven at 60° C. for 5 hours, cut into an average length of 2 mm, and put into an extraction tank (capacity: 30 L). The pine needles were brought into contact with steam for 3 hours while steam was applied thereto at 120° C. and a pressure of 2 atm through a steam generator, thereby obtaining a Pinus Densiflora extract.

Oil positioned at an upper layer portion of the obtained Pinus Densiflora extract was separated, and then allowed to sequentially pass through a granular activated carbon column (manufacturer: Calgon Mitsubishi Chemical Corp., Japan) and a ultrafiltration device (0.2 μm, Ministart, Satorius Stedim Biotech, Germany) to obtain pure oil from which impurities were removed.
Produced amount: 160 g
Yield: 3.2% w/w
Specific gravity: 0.93 g/cm³

### <Example 1-1> Preparation of Test Solution Including Oil Separated from Pinus Densiflora Extract

2 g of polysorbate 80 as a surfactant was added to 100 mL of distilled water, and 1 g of the oil separated from the Pinus densiflora extract obtained according to Example 1 was added to the mixed solution and stirred at 3,000 rpm for 30 minutes, and 400 mL of distilled water was added thereto to adjust the total amount to 500 mL such that the oil separated from the Pinus densiflora extract was included in an amount of 2 mg with respect to 1 mL of the mixed solution, thereby completing the preparation of a test solution.

### <Example 2> Preparation of Oil Separated from Abies Holophylla Maxim Extract

5 kg of fir needles (Collection place: Pyeongchang, Gangwon-do) were taken, washed with 1.5 L of distilled water, dried in a dry oven at 60° C. for 5 hours, cut into an average length of 2 mm, and put into an extraction tank (capacity: 30 L). The fir needles were brought into contact with steam for 3 hours while steam was applied thereto at 120° C. and a pressure of 2 atm through a steam generator, thereby obtaining an Abies Holophylla Maxim extract.

Oil positioned at an upper layer portion of the obtained Abies Holophylla Maxim extract was separated, and then allowed to sequentially pass through a granular activated carbon column (manufacturer: Calgon Mitsubishi Chemical Corp., Japan) and a ultrafiltration device (0.2 μm, Ministart, Satorius Stedim Biotech, Germany) to obtain pure oil from which impurities were removed.
Produced amount: 125 g
Yield: 2.5% w/w
Specific gravity: 0.9 g/cm³

### <Example 2-1> Preparation of Test Solution

2 g of polysorbate 80 as a surfactant was added to 100 mL of distilled water, and 1 g of the oil separated from the Abies holophylla Maxim extract obtained according to Example 2 was added to the mixed solution and stirred at 3,000 rpm for 30 minutes, and 400 mL of distilled water was added thereto to adjust the total amount to 500 mL such that the oil separated from the Abies holophylla Maxim extract was included in an amount of 2 mg with respect to 1 mL of the mixed solution, thereby completing the preparation of a test solution.

Test Methods

<Method 1> Specific Gravity Measurement Method

The specific gravity of each of the oils separated from Pinus densiflora and Abies holophylla Maxim was measured according to the first method (measurement method using a pycnometer) of the specific gravity and density measurement method among general test methods of the Korean Pharmacopoeia eleventh edition.

<Method 2> Setup of Experimental Groups and Test Solution Administration Method

As experimental animals, 6-week-old male Sprague-Dawley (SD) white mice weighing about 250 g were used, and the mice were divided into a normal control, an erectile dysfunction-induced control, and experimental groups in which erectile dysfunction was induced and then the test solutions prepared according to Examples 1-1 and 1-2 were respectively administered, and 10 mice were assigned to each group.

A 20 v/v % aqueous ethanol solution was orally administered daily to the controls and the experimental groups at a dose of 1 mL for 30 days to induce erectile dysfunction due to chronic alcoholism.

In addition, 5 mg/kg of the test solutions of Example 1-1 (test solution including the oil separated from the Pinus densiflora extract) and Example 2-1 (test solution including the oil separated from the Abies holophylla Maxim extract) were respectively administered to the experimental groups once a day for 30 days using a zonde for oral administration (Oral zonde, 15 G, Samwoo Science, Korea) from the date at which the 20 v/v % aqueous ethanol solution started to be administered.

<Method 3> Preparation Method of Enzyme Source

Six-week-old male SD white mice were placed in an anesthesia chamber (JD-C-107R, 245×395×250 mm, Jeung-Do Bio & Plant Co., Ltd., Korea), saturated with ether, and subjected to inhalation anesthesia for 15 minutes, and then the lower abdomen of each mouse was incised about 3 cm, about 7 mL of blood was collected from the abdominal aorta, and then the penile tissue was separated under the pubic bone, peeled off to the entry site, and extracted. The corpus cavernosum was isolated from the extracted penile tissues, washed with physiological saline, and the remaining foreign matter and blood were removed by filter paper.

Subsequently, 0.1 M potassium phosphate buffer (pH 7.4) in a four-fold amount per 1 g of the corpus cavernosum tissue was added thereto, and homogenized using a homogenizer (ULTRA TURRAX®IKA®T18basic) at 6,000 rpm per minute to thereby prepare a ground homogenate.

The ground homogenate was centrifuged at 600×g for 10 minutes using a refrigerated centrifuge (Hanil Union 32R) to collect a supernatant, the supernatant was used as a nitrite content measurement source and centrifuged again at 10,000×g for 30 minutes to collect a supernatant, which was then used as an enzyme source for measuring the activity of nitric oxide synthase.

In addition, a 0.25 M sucrose solution (containing 0.02M Tris HCl buffer (pH 4.0), 1 mM ethylenediamine tetra acetic acid (EDTA), and 10 mM 2-mercaptoethanol) was added in a 5-fold amount per 1 g of the corpus cavernosum tissue and homogenized using a homogenizer (ULTRA TURRAX®IKA®T18basic) at 6,000 rpm per minute to thereby prepare a ground homogenate. The ground homogenate was centrifuged using a refrigerated centrifuge (Hanil Union 32R) at 8,000×g for 20 minutes to collect a supernatant, which was then used as an enzyme source for measuring the activity of guanylate cyclase and a source for measuring the content of cyclic-GMP nitrite.

<Method 4> Nitrite Content Measurement Method

To measure the content of nitrite, 200 μL of a Griess reagent [1% (w/v) sulfanilamide, 5% (v/v) phosphoric acid, and 0.1% (w/v) naphthylethylene diamine dihydrochloride] was added to 200 μL of the enzyme source, a reaction was allowed to occur therebetween at room temperature for 10 minutes, and then absorbance at 550 nm was measured (Genesys 20 Thermo Scientific, USA). The content of nitrite was calculated based on a standard curve of sodium nitrite, and the amount of nitrite per 1 g of tissue was converted to μmole.

<Method 5> Method of Evaluating Nitric Oxide Synthase Activity

Nitric oxide synthase activity was measured by colorimetric assay using a NADPH diaphorase activity measurement method. To 50 μL of the tissue enzyme source of each experimental animal were added a 50 mM 4-(2-hydroxyethyl)-1-piperazine ethane sulfonic acid (HEPES, pH 7.4) solution, 25 μM of L-arginine, 1 mM nicotinamide adenine dinucleotide phosphate (NADPH, reduced form), 2 mM ethylenediamine tetra acetic acid (EDTA), 1.5 mM CaCl₂, 1 mM dithiothreitol, 2 μg of calmodulin, and 1 mM nitroblue tetrazolium (NBT), a reaction was allowed to react therebetween at 37° C. for 5 minutes, and a change in absorbance at 585 nm was measured using Genesys 20 Thermo Scientific. The enzymatic activity was calculated as a value obtained by dividing the absorbance measured at a wavelength of 585 nm using Genesys 20 Thermo Scientific by the content of a used protein.

<Method 6> Method of Measuring Guanyl Cyclase Activity and Cyclic-GMP Content

For the measurement of guanyl cyclase activity, 60 nmole of [3H] guanosine 5′-triphosphate (GTP) (0.02 μCi/nmole), cyclic-GMP (0.3 μmole), manganese chloride (MnCl₂, 0.3 mole), and Tris HCl buffer (pH 7.7, 30 μmole) were added to a standard reaction solution, and then an enzyme solution was added thereto to a final volume of 0.15 mL and mixed well, and a reaction was allowed to occur therebetween at 30° C. for 20 minutes while being stirred at 50 rpm in a thermostatic shaking incubator (BR-300LR, Taitec, Japan). After the reaction, the resulting solution was heated in boiling water for 2 minutes and cooled with iced water to terminate the reaction. The amount of cyclic-GMP produced after the enzymatic reaction was measured and subjected to aluminum oxide column chromatography. The reaction solution was added to a column, and then 5 mL of 0.05 M Tris HCl buffer (pH 7.4) was added thereto to perform perfusion, and a cyclic-GMP-containing perfusate collected through the column was perfused again with a 0.05 N formic acid solution and a 0.2 M ammonium formate solution containing 4 N formic acid through a Dowex 1-X2 column to collect the perfusate, which was then used to measure radioactivity to calculate the content. Radioactivity was measured using a liquid scintillation spec-trometer (Beckman, LS-233). Enzyme activity was determined by measuring radioactivity trapped by cyclic-GMP in a nonradioactive form from [3H] guanosine 5′-triphosphate (GTP) and expressed as cpm. Meanwhile, for the measurement of GMP concentration of the corpus cavernosum tissue, experiments were conducted in the same manner as in the above experiments, and then the unit of content was converted to pmole/mg protein/min.

<Method 7> Method of Evaluating Penile Erectility According to Change in Pressure of Corpus Cavernosum

For anesthesia, pentobarbital sodium was injected into the abdominal cavity of each white mouse at a dose of 3 mg/kg, and then the lower abdomen of each mouse was incised to a size of about 3 cm, about 7 mL of blood was collected from the abdominal aorta, and then the penile tissue was separated under the pubic bone and peeled off to the entry site, and the penile cavernous nerves were isolated therefrom. For neural stimulation, a platinum electrode was installed in the corpus cavernosum nerve and connected to an electrical stimulator (SEN-7103, Nihon Kohden, Japan).

In addition, the penile epithelium was incised to expose the corpus cavernosum, and for the measurement of an intracavernosal pressure, a 26 G needle was placed in the corpus cavernosum, which was then connected to a differential amplifier (DA 100, Biopac System, USA) through Sorenson Transpac cable (Abbott Critical Care System, USA) and the intracavernosal pressure was measured using Data Acquisition (MP 100, Biopac Systems, USA), and measurement values were recorded and analyzed using a data analysis program (Acqknowledge 1.5.5 program, Biopac Systems, USA). Intermittent perfusion was performed with heparinized saline (5,000 IU/ml) to prevent blood coagulation in a pressure transmission pipe.

For penile erectility observation, a penile erection was caused by applying cavernous nerve stimulation to each animal (Frequency: 1 Hz, Intensity: 3-5 V, Pulse duration: 1 msec) for 1 minute to set a standard for a normal penile erection. Thereafter, the intracavernosal pressure was lowered to the baseline, and after 15 minutes, the same intensity of electrical stimulation was applied to measure penile erectility.

<Method 8> Method of Evaluating Penile Erectility Using L-NAME

For the measurement of penile erectility using L-NAME, 10 minutes after the intracavernosal pressure was lowered to the baseline, L-NAME was injected into the corpus cavernosum according to concentration, and after 15 minutes, a penile erection was caused with electrical stimulation to observe penile erectility according to the concentration of a drug.

Hereinafter, the present invention will be described in further detail with reference to the following Experimental Examples. These Experimental Examples are intended to aid in the understanding of implementation of the present invention and are not intended to limit the scope of the present invention.

### Experimental Examples

### <Experimental Example 1> Content Measurement of Nitrite (NO₂)

As a result of content measurement, the nitrite content of the corpus cavernosum tissue of the normal control was 0.57±0.04 μmole/g, whereas the control administered ethanol exhibited a significant decrease in nitrite content, i.e., 0.35±0.06 μmole/g, compared to the normal control, and the experimental groups, i.e., the group administered the oil separated from the Pinus densiflora extract and the group administered the oil separated from the Abies holophylla Maxim extract, exhibited significant increases in nitrite content, i.e., 0.49±0.07 μmole/g and 0.51±0.09 μmole/g, respectively, compared to the control.

### <Experimental Example 2> Measurement of Nitric Oxide Synthase Activity

As a result of conducting a test for the measurement of nitric oxide synthase activity, the nitric oxide synthase activity of the normal control was 1.42±0.18 ΔOD/mg, whereas the control exhibited significantly inhibited nitric oxide synthase activity, i.e., 0.96±0.08 ΔOD/mg, compared to the normal control. In contrast, the experimental groups, i.e., the group administered the oil separated from the Pinus densiflora extract and the group administered the oil separated from the Abies holophylla Maxim extract, exhibited significant increases in nitric oxide synthase activity, i.e., 1.25±0.05 ΔOD/mg and 1.20±0.07 ΔOD/mg, respectively, compared to the control.

<Experimental Example 3> Measurement of Guanyl Cyclase Activity and Cyclic-GMP Content

As a result of conducting the experiment for guanyl cyclase activity, the normal control exhibited a guanyl cyclase activity of 103.7±7.5 cpm, whereas the control exhibited significantly inhibited guanyl cyclase activity, i.e., 77.3±6.2 cpm, compared to the normal control. In contrast, the experimental groups, i.e., the group administered the oil separated from the Pinus densiflora extract and the group administered the oil separated from the Abies holophylla Maxim extract, exhibited significant increases in guanyl cyclase activity, i.e., 93.2±7.1 cpm and 95.3±4.9 cpm, respectively, compared to the control.

As a result of conducting the experiment for cyclic-GMP content, the normal control exhibited a c-GMP content of 0.150±0.008 pmole/mg, whereas the control exhibited a significant decrease in c-GMP content, i.e., 0.082±0.012 pmole/mg, compared to the normal control. However, the experimental groups, i.e., the group administered the oil separated from the Pinus densiflora extract and the group administered the oil separated from the Abies holophylla Maxim extract, exhibited significant increases in c-GMP content, i.e., 0.115±0.006 pmole/mg and 0.125±0.007 pmole/mg, respectively, compared to the control.

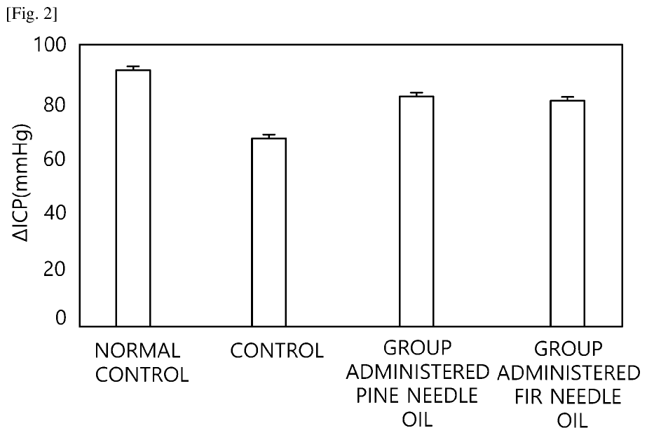

From the result that the experimental groups respectively administered the oil separated from the Pinus densiflora extract and the oil separated from the Abies holophylla Maxim extract exhibited an increase in c-GMP content in the corpus cavernosum smooth muscle, it can be seen that the oil separated from the Pinus densiflora extract and the oil separated from the Abies holophylla Maxim extract affect the generation of nitric oxide (see FIG. 2).

### <Experimental Example 4> Penile Erectility Evaluation

The internal pressure of the corpus cavernosum of the normal control was 90.9±7.8 mmHg, whereas the control administered an ethanol solution exhibited a significant decrease in the internal pressure thereof, i.e., 66.7±4.3 mmHg, compared to the normal control. In contrast, the respective experimental groups administered the oil separated from the Pinus densiflora extract and the oil separated from the Abies holophylla Maxim extract while ethanol was administered exhibited significant increases in the internal pressure of the corpus cavernosum, i.e., 82.1±4.8 mmHg and 80.1±5.2 mmHg, respectively, compared to the control.

### <Experimental Example 5> Changes in Nitric Oxide Content and c-GMP Content after L-NAME Treatment

In the present invention, the term “L-NAME” is an abbreviation of N′-nitro-L-arginine-methyl ester hydrochloride and refers to a compound which is rep-resented by Formula 1 below, has the molecular formula of C₇H₁₆ClN₅O₄ (number average molecular weight: 269.69 g/mol), and functions as a nitric oxide synthase inhibitor.

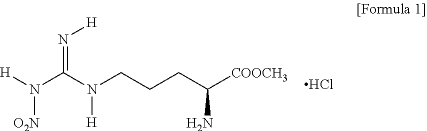 [SMILES: [H]/N=C(\NCCC[C@H](N)C(=O)OC)N[N+](=O)[O-] ; Cl]

After L-NAME treatment, the nitric oxide content of the corpus cavernosum tissue was evaluated.

Specifically, normalization treatment to lower the intracavernosal pressure of each white mouse to the baseline was performed, and 20 minutes after L-NAME was injected into the corpus cavernosum according to concentration, the penile cavernous tissues were extracted, and the nitric oxide content was observed. The content of nitric oxide in the corpus cavernosum tissues of the normal control was 0.56±0.04 μmole, but as the amount of injected L-NAME increased, the content of nitric oxide decreased, i.e., 0.51±0.08 μmole at the concentration of injected L-NAME of 10⁻⁷ M, 0.48±0.05 mole at 10⁶M, and 0.41±0.03 mole at 10⁵M.

In addition, after the normalization treatment to lower the intracavernosal pressure of each white mouse to the baseline, the cyclic-GMP content of the corpus cavernosum tissue was 0.106±0.005 pmole/mg. When observing the content of cyclic-GMP after L-NAME was injected at various concentrations under the same conditions, the content of cyclic GMP decreased as the amount of L-NAME injected into the corpus cavernosum increased, i.e., 0.097±0.008 pmole/mg at 10⁷M, 0.092±0.005 pmole/mgat10⁶M, and 0.085±0.004 pmole/mgat10⁵M.

### <Experimental Example 6> Evaluation of Penile Erectility by L-NAME Pre-treatment

The intracavernous pressure of the normal control was 90.9±7.8 mmHg, and was significantly reduced when treated with L-NAME, such as 89.9±6.5 mmHg at a concentration of L-NAME of 10⁷M, 86.6±8.3 mmHg at 10⁶M, 84.2±8.8 mmHg at 10⁵M, and 70.8±6.6 mmHg at 10⁴M. However, the intracavernous pressures of the group administered the oil separated from the Pinus densiflora extract and the group administered the oil separated from the Abies holophylla Maxim extract were 82.5±8.8 mmHg at a concentration of injected L-NAME of 10⁴M and 86.6±4.7 mmHg at a concentration of injected L-NAME of 10⁴M, respectively, showing restoration to almost anormal level.

Hereinafter, the present invention will be described in further detail with reference to the following Preparation Examples. These Preparation Examples are provided to aid in understanding of the practice of the present invention and are not intended to limit the scope of the present invention.

### Preparation Examples

### <Preparation Example 1> Preparation of Liquid

An inclusion compound was prepared by physically kneading β-cyclodextrin and the oil separated from a Pinus densiflora extract. Specifically, hydroxypropyl β-cyclodextrin was added to a reactor, and the oil separated from the Pinus densiflora extract was added thereto, followed by mixing and stirring for 30 minutes using mortars to thereby prepare an inclusion compound of the oil separated from the Pinus densiflora extract and hydroxypropyl β-cyclodextrin.

Subsequently, 60 mL of distilled water was added to the inclusion compound to dissolve the compound therein, xylitol and cherry flavor powder were added thereto, the resulting solution was stirred at 3,000 rpm for 20 minutes using a homomixer, thereby preparing a total amount of 100 mL of a liquid containing the oil separated from the Pinus densiflora extract. Used raw materials are the same as shown in Table 1 below.

TABLE 1

| Raw materials | Added amount | |
|---|---|---|
| Oil seperated from Pinus densiflora extrtact | 0.3 | g |
| Hydroxypropyl β-cyclodextrin | 3 | g |
| Xylitol | 30 | g |
| Cherry flavor powder | 0.05 | g |
| Distilled water | 75 | mL |

### <Preparation Example 2> Preparation of Liquid

A liquid containing the oil separated from the Abies holophylla Maxim extract was prepared using the same method as that used in Preparation Example 1, except that 0.3 g of the oil separated from the Abies holophylla Maxim extract was added instead of the oil separated from the Pinus densiflora extract.

### <Preparation Example 3> Preparation of Granules

Hydroxypropyl β-cyclodextrin was added to a reactor, and the oil separated from the Pinus densiflora extract was added thereto, followed by stirring at 1,000 rpm for 30 minutes using a mixer to thereby prepare an inclusion compound of the oil separated from the Pinus densiflora extract and hydroxypropyl β-cyclodextrin.

Separately, dextrose, lactose, and microcrystalline cellulose were added to a speed mixer, and the inclusion compound of the oil separated from Pinus densiflora extract and hydroxypropyl β-cyclodextrin was added thereto, and 100 mL of distilled water was added thereto, followed by stirring at 800 rpm for about 1 hour. The stirred mixture was dried in a dryer at 70° C. for 5 hours, and then magnesium stearate was added thereto to thereby prepare granules containing the oil separated from the Pinus densiflora extract through a granulator. Used raw materials are the same as shown in Table 2 below.

TABLE 2

| Raw materials | Added amount | |
|---|---|---|
| Oil seperated from Pinus densiflora extrtact | 30 | g |
| Hydroxypropyl β-cyclodextrin | 300 | g |
| Dextrose | 200 | g |
| Lactose | 200 | g g |
| Microcrystalline cellulose | 300 | g |
| Magnesium stearate | 2 | g |
| Distilled water | 100 | mL |

### <Preparation Example 4> Preparation of Granules

Granules containing the oil separated from the Abies holophylla Maxim extract was prepared using the same method as that used in Preparation Example 3, except that 30 g of the oil separated from the Abies holophylla Maxim extract was added instead of the oil separated from the Pinus densiflora extract.

### <Preparation Example 5> Preparation of Chewable Tablets

The oil separated from the Pinus densiflora extract and β-cyclodextrin were added to a mixer and stirred at 1,000 rpm for 30 minutes to thereby prepare an inclusion compound of the oil separated from the Pinus densiflora extract and hydroxypropyl β-cyclodextrin.

Separately, maltodextrose, xylitol, and microcrystalline cellulose were added to a speed mixer, and the inclusion compound of the oil isolated from Pinus densiflora extract and hydroxypropyl β-cyclodextrin was added thereto, and 130 mL of distilled water was added thereto, followed by stirring at 800 rpm for about 1 hour. The stirred mixture was dried in dryer at 70° C. for 5 hours and granulated in a granulator, and then magnesium stearate and yogurt flavor powder were added thereto and mixed to thereby prepare 1,000 mg/tablet of chewable tablets through a tablet press. Used raw materials are the same as shown in Table 3 below.

TABLE 3

| Raw materials | Added amount | |
|---|---|---|
| Oil seperated from Pinus densiflora extrtact | 40 | g |
| Hydroxypropyl β-cyclodextrin | 400 | g |
| Maltodextrin | 100 | g |
| Microcrystalline cellulose | 500 | g |
| Xylitol | 300 | g |
| Yogurt flavor powder | 3 | g |
| Magnesium stearate | 3 | g |
| Distilled water | 130 | mL |

### <Preparation Example 6> Preparation of Chewable Tablets

Chewable tablets containing the oil separated from the Abies holophylla Maxim extract was prepared using the same method as that used in Preparation Example 5, except that 40 g of the oil separated from the Abies holophylla Maxim extract was added instead of the oil separated from the Pinus densiflora extract.

1. A pharmaceutical composition for preventing or treating male sexual dysfunction, the pharmaceutical composition comprising, as an active ingredient, oil separated from a Pinus densiflora extract, oil separated from an Abies holophylla Maxim extract, or a mixture thereof.

2. The pharmaceutical composition of claim 1, wherein the oil is obtained from a pine needle extract or a fir needle extract.

3. The pharmaceutical composition of claim 1, wherein the composition comprises a pharmaceutically acceptable carrier.

4. The pharmaceutical composition of claim 1, wherein the composition is for oral administration.

5. The pharmaceutical composition of claim 1, wherein the oil is included in an amount of 0.1 wt % to 15 wt % with respect to a total weight of the composition.

6. The pharmaceutical composition of claim 1, wherein the extract is obtained by subjecting pine needles or fir needles to steam distillation.

7. The pharmaceutical composition of claim 1, wherein the male sexual dysfunction is caused by alcohol ingestion.

8. The pharmaceutical composition of claim 1, wherein the oil is obtained by the following processes:
(S1) collecting pine needles, fir needles, or a mixture thereof; and
(S2) contacting the collected pine needles, the collected fir needles, or the collected mixture thereof with steam to obtain an extract thereof.

9. The pharmaceutical composition of claim 8, wherein the contacting comprises injecting steam at 1 atm to 8 atm into a reactor comprising pine needles, fir needles, or a mixture thereof.

10. The pharmaceutical composition of claim 8, further comprising separating oil from the obtained extract.

11. The pharmaceutical composition of claim 1, wherein the male sexual dysfunction comprises erectile dysfunction, premature ejaculation, or prostatic hyperplasia.

12. The pharmaceutical composition of claim 1, wherein the composition is in the form of a liquid, a suspension, powder, granules, a tablet, a capsule, a pill, or an extract.

13. A health functional food for preventing or treating male sexual dysfunction, the health functional food comprising, as an active ingredient, oil separated from a Pinus densiflora extract, oil separated from an Abies holophylla Maxim extract, or a mixture thereof.

14. A formulation comprising: oil separated from a Pinus densiflora extract, oil separated from an Abies holophylla Maxim extract, or a mixture thereof; and β-cyclodextrin.

15. The formulation of claim 14, wherein the β-cyclodextrin comprises any one of 2,6-dimethyl-β-cyclodextrin, 2-hydroxyethyl-β-cyclodextrin, and 2-hydroxypropyl-β-cyclodextrin.

16. The formulation of claim 14, wherein the formulation comprises a β-cyclodextrin inclusion compound.

17. The formulation of claim 16, wherein the β-cyclodextrin inclusion compound is a compound in which oil separated from a Pinus densiflora extract, oil separated from an Abies holophylla Maxim extract, or a mixture thereof is encapsulated in an internal cavity of β-cyclodextrin.

18. A health functional food comprising: oil separated from a Pinus densiflora extract, oil separated from an Abies holophylla Maxim extract, or a mixture thereof; and β-cyclodextrin.
